(19) United States
(12) Patent Application Publication
(10) Pub. No.: US 2010/0278925 A1
(43) Pub. Date: Nov. 4, 2010

(54) FORMULATIONS OF ORGANO-PLATINIC COMPOUNDS IN THE PRESENCE OF ASSOCIATIVE POLYMERS, PRODUCTS THUS OBTAINED AND USES THEREOF

(75) Inventors: Olivier Guerret, La Tour De Salvagny (FR); Jean-Marc Suau, Lucenay (FR)

Correspondence Address: OBLON, SPIVAK, MCCLELLAND MAIER & NEUSTADT, L.L.P., 1940 DUKE STREET, ALEXANDRIA, VA, 22314, US

(73) Assignee: Coatex S.A. S., Genay (FR)

(21) Appl. No.: 12/738,302

(22) PCT Filed: Oct. 7, 2008
(86) PCT No.: PCT/IB08/02669

§ 371 Date: Jun. 22, 2010

(30) Foreign Application Priority Data
Oct. 19, 2007 (FR) 07/07306

(51) Int. Cl. A61K 9/50; A61K 33/24; A61K 31/282; A61K 31/337; A61K 38/08; A61K 31/513; A61P 35/00

(52) U.S. Cl. 424/494; 424/497; 424/649; 514/492; 514/449; 514/19.3; 514/274

(57) ABSTRACT

The invention consists of formulations based on compounds of platinum encapsulated by associative and water-soluble polymers. These formulations are in aqueous form or in the form of granulates. The invention further pertains to pharmaceutical preparations which contain these formulations, and their implementation in the fabrication of an orally administered medication, in polychemotherapy treatments.

The invention pertains to a new type of formulation of organoplatinic compounds (organic derivatives of platinum) to be administered orally.

Among organoplatinic compounds, cisplatin and oxaliplatin are platinum-based molecules used in the treatment of various cancers such as sarcomas, carcinomas (small-cell lung cancer, ovarian cancer etc.), and lymphomas. They belong to the class of compounds alkylating DNA with carboplatin.

However, these compounds cause numerous unwanted effects, particularly when administered intravenously: gastrointestinal nephrotoxicity (nausea, vomiting), neurotoxicity, and moderate myelosuppression. Work has been done in view of testing analogues for these active ingredients, but none has proven as effective as the basic molecules. Studies have also been carried out in view of combining other therapies, with reduced quantities of cisplatin and oxyplatin, but this has reduced the effectiveness of the treatment.

Another field of research pertains to the methods for administering organoplatinic compounds orally. In this case, the challenge consists of engineering a method for limiting the toxicity of the active ingredients, enabling them to cross the gastrointestinal barrier, to control their release along their path, and finally to offer the patient the option of oral treatment that he or she can administer him- or herself.

Along this line, French Patent Application FR 2,759 293 describes a fairly complex method for fabricating microgranules containing cisplatin. It includes a step of adhering cisplatin onto microgranules by spraying an organic solution containing cisplatin onto a neutral substrate, then a second step of coating with the help of polymers that enable the prolonged release of the active ingredient. Coated substances are chosen from among cellulosic or methacrylic polymers, and preferentially from among methacrylic acid copolymers with an acrylic ester sold under the brand name Eudragit™. This coating constitutes a varnish that protects the active ingredient, which will gradually dissolve in an aqueous medium: thus, the release of the organoplatinic compound continuously.

Continuing her research into enriching the state of the art with an alternative solution that makes it possible to administer organoplatinic compounds orally, while protecting the patient from their toxicity, and enabling passage through the gastrointestinal barrier and controlling the gradual release of the active ingredient, the Applicant has engineered new formulations, which contain at least one organoplatinic compound and at least one associative water-soluble polymer, which formulations are characterized in that said associative water-soluble polymer is the result of the polymerization:
of (meth)acrylic acid,
of at least one non-water-soluble monomer, which is preferentially a (meth)acrylic ester chosen very preferentially from among ethyl acrylate, butyl acrylate, methyl methacrylate, and mixtures thereof,
and of at least one monomer, of formula (I):

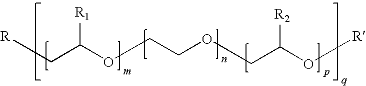

m, n, p and q are whole numbers and m, n, p are less than 150 and at least one of them is greater than 0, preferentially 20,
R has a polymerizable vinylic function,
R₁ and R₂ are identical or different, and represent hydrogen atoms or alkyl groups having 1 to 4 carbon atoms,
R′ is a hydrophobic group having 14 inclusive to 32 inclusive carbon atoms, and is preferentially chosen from among branched alkyl groups having 14 inclusive to 32 inclusive carbon atoms, and from among substituted aromatic groups having from 14 inclusive to 32 inclusive carbon atoms.

The associative water-soluble polymers described above are also known as HASE polymers (standing for Hydrophobically Alkali Swellable Emulsion): they refer to acrylic polymers based on (meth)acrylic acid, a non-water-soluble monomer which is preferentially a (meth)acrylic ester, and an associative hydrophobic monomer. They are distinct from ASE (Alkali Soluble Emulsion) polymers such as Eudragit™ in the data they contain a hydrophobic monomer that may develop associative interactions under certain pH conditions.

This monomer has the property, in an aqueous environment and at a high pH, of developing associative interactions which lead to the formation of nanometric nodules (diameter between 0.1 and 100 nm): these act as solvation cages with respect to the organoplatinic compounds. The active ingredient, isolated in this manner, will be gradually released through the nodules created by the polymer. The patient is therefore protected, and the efficiency of the treatment is improved.

One of the Applicant's merits lies in having learned how to use this associative mechanism to insulate organoplatinic compounds. Another one of her merits is having learned how to select and engineer the particular hydrophobic monomers which make it possible to effectively insulate the organoplatinic compounds: the selection particularly relies on the length and nature of the R′ chain as indicated above, and is illustrated in the examples of the present Application.

In a first variant, the inventive formulations are aqueous formulations which contain the organoplatinic compound and the associative water-soluble polymer; figure obtained by mixing the various components, and regulating the pH to a value greater than 6, so as to trigger the associative effect caused by the monomer with formula (I). Practically speaking, the pH is adjusted by means of a mineral or organic base, and the components (organoplatinic compound, water, polymer, as well as the mineral or organic base) are introduced into the reactor while being agitated. This variant corresponds to the liquid form of product that the patient can ingest: that of a syrup.

In a second variant, the pH of the formulations previously obtained is levered to a value below 6, preferentially 3, for example by means of a medium-strong to strong acid: by means of a precipitation mechanism, the structure of the nodules “collapses” onto the organoplatinic compounds. Thanks to a later step of removing the water, such as through drying or filtration, dry formulations are obtained which contain the organoplatinic compound and the associative polymer. This variant corresponds to the dry form of product that the patient can swallow: that of a granulate.

Thus, another benefit of the invention resides in the existence of these two variants, which each lead to a particular form of the final product, which are precisely the two forms sought by the galenist. Furthermore, the unity of the invention is ensured between these two forms by the presence, in both, of the associative water-soluble polymer and the organoplatinic compound.

A first object of the invention therefore consists of aqueous formulations, containing at least one organoplatinic compound at least one associative water-soluble polymer, and characterized in that said polymer is result of the polymerization of:
of (meth)acrylic acid,
of at least one non-water-soluble monomer, which is preferentially a (meth)acrylic ester chosen very preferentially from among ethyl acrylate, butyl acrylate, methyl methacrylate, and mixtures thereof,
and of at least one monomer, of formula (I):

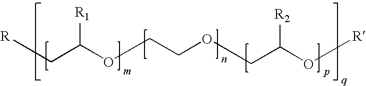

m, n, p and q are whole numbers and m, n, p are less than 150 and at least one of them is greater than 0, preferentially 20,
R has a polymerizable vinylic function,
R₁ and R₂ are identical or different, and represent hydrogen atoms or alkyl groups having 1 to 4 carbon atoms,
R′ is a hydrophobic group having 14 inclusive to 32 inclusive carbon atoms, and is preferentially chosen from among branched alkyl groups having 14 inclusive to 32 inclusive carbon atoms, and from among substituted aromatic groups having from 14 inclusive to 32 inclusive carbon atoms.

It is understood that these 3 monomers are essential in forming the inventive associative water-soluble polymers. The person skilled in the art may add other monomers, such as, for example, a monomer possessing at least two ethlenically unsaturated functions, also known as a crosslinking monomer.

In a first variant, these aqueous formulations are further characterized in that their pH is greater than 6, preferentially 6.5, and very preferentially 7.

In a second variant, these aqueous formulations are further characterized in that their pH is less than 6, preferentially 3. It is obvious that this variant is obtained by reducing the pH, the pH having previously been increased to a value greater than 6 so as to trigger the associative effect that makes it possible to protect the organoplatinic compound.

These aqueous formulations are further characterized in that they contain from 0.1 to 30%, preferentially from 5% to 30%, and very preferentially from 10% to 30% by dry weight of said organoplatinic compound, in relation to the dry weight of said associated water-soluble polymer.

These aqueous formulations are further characterized in that they contain from 0.1% to 15%, and preferentially from 1% to 10% by weight of solids in relation to their total weight.

These aqueous formulations are further characterized in that the organoplatinic compound is chosen from among cisplatin, carboplatin, oxaliplatin and mixtures thereof, and is preferentially oxaliplatin.

These aqueous formulations are further characterized in that they potentially contain at least one other anticancer agent chosen from among fluoro-uracil, S1, the association of vinblastin with bleomycin, the association of etoposide with bleomycin, or paclitaxel.

A further object of the invention consists of granulates, containing at least one organoplatinic compound at least one associative water-soluble polymer, and characterized in that said polymer is result of the polymerization:
of (meth)acrylic acid,
of at least one non-water-soluble monomer, which is preferentially a (meth)acrylic ester chosen very preferentially from among ethyl acrylate, butyl acrylate, methyl methacrylate, and mixtures thereof,
and of at least one monomer, of formula (I):

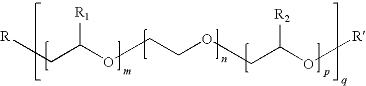

m, n, p and q are whole numbers and m, n, p are less than 150 and at least one of them is greater than 0, preferentially 20,
R has a polymerizable vinylic function,
R₁ and R₂ are identical or different, and represent hydrogen atoms or alkyl groups having 1 to 4 carbon atoms,
R′ is a hydrophobic group having 14 inclusive to 32 inclusive carbon atoms, and is preferentially chosen from among branched alkyl groups having 14 inclusive to 32 inclusive carbon atoms, and from among substituted aromatic groups having from 14 inclusive to 32 inclusive carbon atoms.

These granulates are further characterized in that they contain less than 5%, preferentially 1%, and a very preferentially 0.1% by weight of water, as measured by a differential scale after evaporation in an oven at 110° C. for 1 hour.

These granulates are further characterized in that they contain from 0.1 to 30%, preferentially from 5% to 30%, and very preferentially from 10% to 30% by dry weight of said organoplatinic compound, in relation to the dry weight of said associated water-soluble polymer.

These granulates are further characterized in that the organoplatinic compound is chosen from among cisplatin, carboplatin, oxaliplatin and mixtures thereof, and is preferentially oxaliplatin.

These granulates are further characterized in that they potentially contain at least one other anticancer agent chosen from among fluoro-uracil, S1, the association of vinblastin with bleomycin, the association of etoposide with bleomycin, or paclitaxel.

These granulates are further characterized in that they contain a coating agent which is chosen from among a cellulosic polymer, a copolymer of (meth)acrylic acid with an acrylic ester, and mixtures thereof.

These granulates are further characterized in that they contain a lubricating agent which is preferentially talc.

These granulates are further characterized in that they contain a plastifying agent which is preferentially triethyl citrate.

These granulates are further characterized in that they contain a surface-active agent which is preferentially polysorbate 80 (also known by the name Tween™ 80, and sold by the company UNIQEMAT™).

A further object of the invention is a pharmaceutical preparation characterized in that it contains an aqueous formulation according to the invention or a granulate according to the invention.

A final object of the invention is the use of an aqueous formulation according to the invention and a granulate according to the invention to manufacture an orally administered medication intended to be used in polychemotherapy.

### EXAMPLES

### Example 1

### Synthesis of Monomers Implemented According to the Invention

### Protocol a: Synthesis of Methacrylic Monomer:

In a 1-liter reactor, the following is weighed out:
400 grams of condensed behenic alcohol with 25 moles of melted ethylene oxide,
0.0994 grams of alloocimene,
43.75 grams of methacrylic anhydride.

The mixture is heated to 82° C.±2° C. and cooking is continued for 3 hours at this temperature. The resulting macromonomer is then diluted with 396 g of methacrylic acid in order to achieve a solution that is liquid at room temperature.

### Protocol b: Synthesis of Urethane Monomer:

In a first step, a pre-condensate is created by weighing into an Erlenmeyer flask:
13.726 grams of toluene di-isocyanate,
36.1 grams of ethyl acrylate,
0.077 grams of alloocimene,
0.198 grams of dibutyl tin dilaurate.

Next, 10.257 grams of glycol ethylene methacrylate and 10 grams of ethyl acrylate are weighed into a pouring funnel. The contents are poured from this funnel into the flask over 20 minutes at a temperature below 35° C., and are left to react for 30 minutes

In a second step, the condensation is created by weighing 132 g of condensed tri-styryl phenol with 25 moles of ethylene oxide into a 1000 mL reactor, which is kept melted at 65° C. Next, the pre-condensate is poured for 20 minutes at 65° C. onto this alcohol, then cooked for 2 hours at 65° C. Finally, the mixture is diluted with 15.5 grams of ethyl acrylate and 84.6 grams of bipermuted water in order to obtain a liquid at room temperature.

### Protocol c: Synthesis of Hemimaleate Monomer:

In a 1-liter reactor, the following is weighed out:
400 grams of branched alcohol with 32 carbon atoms condensed with 25 moles of melted ethylene oxide,
0.0994 grams of alloocimene,
25.3 grams of maleic anhydride.

The mixture is heated to 82° C.±2° C. and cooking is continued for 3 hours at this temperature. The resulting macromonomer is then diluted with 425 g of methacrylic acid in order to achieve a solution that is liquid at room temperature.

### Example 2

### Synthesis of Associative Water-Soluble Polymers

### Protocol A:

In a 1-liter reactor, 280 grams of bipermuted water and 3.89 grams of sodium dodecyl sulfate are weighed out. The synthesis reactor is heated at the base to 82° C.±2° C.

During this time, a pre-emulsion is prepared, by weighing into a beaker:
112.4 grams of bipermuted water,
2.1 grams of sodium dodecyl sulfate,
80.6 grams of methacrylic acid,
146.1 grams of ethyl acrylate,
55.6 grams of a macromonomer solution as described in protocol a).

Next, 0.85 grams of ammonium persulfate diluted into 10 grams of bipermuted water for the first catalyzer, and 0.085 grams of sodium metabisulfite diluted into 10 grams of bipermuted water for the second catalyzer. When the synthesis reactor's base is at the right temperature, the 2 catalyzers are added, and polymerization is performed for 2 hours at 76° C.±2° C., adding the pre-emulsion in parallel. The pump is rinsed with 20 grams of bipermuted water, and it is cooked for 1 hour at 76° C.±2° C. Finally, the mixture is cooled to room temperature and the polymer thereby obtained is filtered.

### Protocol B:

In a 1-liter reactor, 280 grams of bipermuted water and 3.89 grams of sodium dodecyl sulfate are weighed out. The synthesis reactor is heated at the base to 82° C.±2° C.

During this time, a pre-emulsion is prepared, by weighing into a beaker:
334 grams of bipermuted water,
3.89 grams of sodium dodecyl sulfate,
0.92 grams of dodecyl mercaptan,
80.6 grams of methacrylic acid,
160.55 grams of ethyl acrylate,
60.4 grams of the methylacrylurethane solution described in protocol b).

Next, 0.33 grams of ammonium persulfate diluted into 10 grams of bipermuted water for the first catalyzer, and 0.28 grams of sodium metabisulfite diluted into 10 grams of bipermuted water for the second catalyzer. When the synthesis reactor's base is at the right temperature, the 2 catalyzers are added, and polymerization is performed for 2 hours at 84° C.±2° C., adding the pre-emulsion in parallel. The pump is rinsed with 20 grams of bipermuted water, and it is cooked for 1 hour at 84° C.±2° C. Next, the mixture is cooled to room temperature and filtered.

### Protocol C:

This protocol is the same as protocol B, except that here, the dodecyl mercaptan is removed from the first step of weighing.

### Protocol D:

This protocol is the same as protocol A, except that 0.9 grams of dodecyl mercaptan are added during the initial step of weighing into the beaker.

### Example 3

### Fabrication of Aqueous Formulations of Associative Water-Soluble Polymers in the Presence of Oxaliplatin, at a pH Above 6.5

In all of the tests that follow, oxaliplatin is the product of the same name, sold by the company MIDAS PHARMA™.

### First Reference Test:

This test is a reference which consists of adding the oxaliplatin into water, without the associative water-soluble polymer.

0.09 g of oxaliplatin are added to 8.6 grams of water, the pH being adjusted to a value equal to 7 by means of a 5% solution of sodium hydroxide. The concentration of oxaliplatin in the water is here equal to 10.5 g/L, whereas the solubility of the oxaliplatin is less than 7.9 grams per litre of water.

The result is a powder suspended in the water: for the concentration being studied, the oxaliplatin is dispersed, but it is only partially water-soluble.

### Second Reference Test:

This test is a reference which consists of adding the oxaliplatin into water, along with the associative water-soluble polymer.

0.09 g of oxaliplatin are added to 8.6 grams of water, the pH being adjusted to a value equal to 8 by means of a 5% solution of sodium hydroxide. The concentration of oxaliplatin in the water is here equal to 10.5 g/L, whereas the solubility of the oxaliplatin is less than 7.9 grams per liter of water.

Next, 1.9 g of a water-soluble polymer with 30% solids content, said polymer being made up 36.9% by weight of methacrylic acid and 63.1% by weight of ethyl acrylate.

The result is a powder suspended in the water: the polymer did not successfully encapsulate the oxaliplatin.

### Tests #1 to 12:

These tests illustrate aqueous formulations containing oxaliplatin and associative water-soluble polymers possessing:
a (meth)acrylic monomer,
a non-water-soluble monomer,
and an associative hydrophobic monomer.

Exactly 1.9 g of an emulsion of associative water-soluble polymer with 30% solids content is weighed out. 8.6 grams of a bipermuted water solution are added, and finally at least 100 mg of oxaliplatin, which is a minimal concentration of 0.86% oxaliplatin, is added to the formulation. The medium is agitated during this addition, and the pH is set to 8 using a 5% sodium hydroxide solution.

### Table 1 Shows:

the % by weighed dry weight of oxaliplatin in relation to the dry weight of the associative polymer being implemented,
the percentage in weight of solids content out of the formulation created (this calculation takes into account the quantity of sodium hydroxide added to the medium to set the pH to 8),
the appearance of the resulting formulation.

TABLE 1

| | | % solids content/ | |
|---|---|---|---|
| | % oxaliplatin/ | formulation | Appearance of |
| Test no. | dry polymer | total | the formulation |
| 1 | 14.8 | 5.2 | Powder dispersed |
| | | | in the water |
| 2 | 14.8 | 5.2 | Powder dispersed |
| | | | in the water |
| 3 | 16.3 | 5.4 | Solution |
| 4 | 13.2 | 5.8 | Solution |
| 5 | 13.6 | 5.7 | Solution |
| 6 | 14.5 | 5.8 | Solution |
| 7 | 14.7 | 5.8 | Solution |
| 8 | 13.8 | 5.8 | Solution |
| 9 | 14.4 | 5.7 | Solution |
| 10 | 15.0 | 5.8 | Solution |
| 11 | 15.0 | 5.8 | Solution |
| 12 | 13.5 | 5.7 | Solution |

In each of the tests #1 to 12, we refer to protocols A, B, or C for synthesizing the polymer: depending on the target monomer ratios, the person skilled in the art calculates the weights of the various components to be weighed in each of these protocols. Likewise, we refer to protocols a, b, or c for synthesizing the monomer with formula (I) and the nature and mass of the alcohol used is specified.

Test 1 implements an associative water-soluble polymer, characterized in that it is obtained by a reaction (according to protocol B) between:
36.5% by weight of methacrylic acid,
54.1% by weight of ethyl acrylate,
9.4% by weight of a monomer with formula (I) (according to protocol b) into which is weighed 100 grams of lauric alcohol condensed with 25 moles of ethylene oxide) with R, which is the result of condensation between ethylene glycol methacrylate and toluene diisocyanate, m=p=0, q=1, n=25 and R′ is an alkyl group containing 12 carbon atoms.

Test 2 implements an associative water-soluble polymer, characterized in that it is obtained by a reaction (according to protocol A) between:
36.0% by weight of methacrylic acid,
54.5% by weight of ethyl acrylate,
9.5% by weight of a monomer with formula (I) (according to protocol a) into which is weighed 346.6 grams of dodecylic alcohol condensed with 25 moles of ethylene oxide) with R which is methacrylate, m=p=0, q=1, n=25 and R′ is an alkyl group containing 10 carbon atoms.

Test 3 implements an associative water-soluble polymer, characterized in that it is obtained by a reaction (according to protocol A) between:
36.7% by weight of methacrylic acid,
53.1% by weight of ethyl acrylate,
10.2% by weight of a monomer with formula (I) (according to protocol a) with R which is methacrylate, m=p=0, q=1, n=25 and R′ is an alkyl group containing 22 carbon atoms.

Test 4 implements an associative water-soluble polymer, characterized in that it is obtained by a reaction (according to protocol C) between:
38.9% by weight of methacrylic acid,
53.1% by weight of ethyl acrylate,
8.0% by weight of a monomer with formula (I) (according to protocol b) with R which is the result of condensation between ethylene glycol methacrylate and toluene diisocyanate, m=p=0, q=1, n=25 and R is tristyryl-phenyl group containing 30 carbon atoms.

Test 5 implements an associative water-soluble polymer, characterized in that it is obtained by a reaction (according to protocol B) between:
40.3% by weight of methacrylic acid,
54.7% by weight of ethyl acrylate,
8.0% by weight of a monomer with formula (I) (according to protocol b) into which is weighed 190.1 grams of nonylphenol having 15 carbon atoms and condensed with 50 moles of ethylene oxide) with R which is the result of condensation between ethylene glycol methacrylate and toluene diisocyanate, m=p=0, q=1, n=50 and R is a nonylphenol group containing 15 carbon atoms.

Test 6 implements an associative water-soluble polymer, characterized in that it is obtained by a reaction (according to protocol A) between:
37.3% by weight of methacrylic acid,
54.8% by weight of ethyl acrylate,
7.9% by weight of a monomer with formula (I) (according to protocol a) into which is weighed 439.8 grams of branched alcohol having 32 carbon atoms and condensed with 50 moles of ethylene oxide) with R which is methacrylate, m=p=0, q=1, n=25 and R which is a branched alkyl group containing 32 carbon atoms.

Test 7 implements an associative water-soluble polymer, characterized in that it is obtained by a reaction (according to protocol A) between:
37.4% by weight of methacrylic acid,
55.0% by weight of ethyl acrylate,
7.6% by weight of a monomer with formula (I) (according to protocol a) into which is weighed 627 grams of branched alcohol having 32 carbon atoms and condensed with 40 moles of ethylene oxide) with R which is methacrylate, m=p=0, q=1, n=40 and R which is a branched alkyl group containing 32 carbon atoms.

Test 8 implements an associative water-soluble polymer, characterized in that it is obtained by a reaction (according to protocol A) between:
34.6% by weight of methacrylic acid,
57.7% by weight of ethyl acrylate,
7.7% by weight of a monomer with formula (I) (according to protocol a) with R which is methacrylate, m=p=0, q=1, n=25 and R′ is an alkyl group containing 32 carbon atoms.

Test 9 implements an associative water-soluble polymer, characterized in that it is obtained by a reaction (according to protocol A) between:
37.4% by weight of methacrylic acid,
54.8% by weight of ethyl acrylate,
8.3% by weight of a monomer with formula (I) (according to protocol a) into which is weighed 376.1 grams of branched alcohol having 16 carbon atoms and condensed with 25 moles of ethylene oxide) with R which is methacrylate, m=p=0, q=1, n=25 and R which is a branched alkyl group containing 16 carbon atoms.

Test 10 implements an associative water-soluble polymer, characterized in that it is obtained by a reaction (according to protocol C) between:
40.3% by weight of methacrylic acid,
54.7% by weight of ethyl acrylate,
5.0% by weight of a monomer with formula (I) (according to protocol b) into which is weighed 190.1 grams of nonylphenol having 15 carbon atoms and condensed with 50 moles of ethylene oxide) with R which is the result of condensation between ethylene glycol methacrylate and toluene diisocyanate, m=p=0, q=1, n=50 and R is a nonylphenol group containing 15 carbon atoms.

Test 11 implements an associative water-soluble polymer, characterized in that it is obtained by a reaction (according to protocol A) between:
36.05% by weight of methacrylic acid,
53.65% by weight of ethyl acrylate,
10.3% by weight of a monomer with formula (I) (according to protocol a) into which is weighed 389.8 grams of branched alcohol having 20 carbon atoms and condensed with 25 moles of ethylene oxide) with R which is methacrylate, m=p=0, q=1, n=25 and R which is a branched alkyl group containing 20 carbon atoms.

Test 12 implements an associative water-soluble polymer, characterized in that it is obtained by a reaction (according to protocol A) between:
33.7% by weight of methacrylic acid,
59.0% by weight of ethyl acrylate,
7.3% by weight of a monomer with formula (I) (according to protocol a) into which is weighed 376.1 grams of branched alcohol having 16 carbon atoms and condensed with 25 moles of ethylene oxide) with R which is methacrylate, m=p=0, q=1, n=25 and R which is a branched alkyl group containing 16 carbon atoms.

Only tests 3 to 12 illustrate the invention, and they correspond to the particular choice of the monomer (I), which has lead to the creation of solutions: here, oxaliplatin has successfully been encapsulated.

### Example 4

### Fabricating Granulates Based on Associative Water-Soluble Polymers and Oxaliplatin

In this example, some of the aqueous formulations which were used to obtain a solution in example 3 were reused (the same test number was kept).

The pH of said formulations was lowered to 5.8 by adding a 4% solution of phosphoric acid.

The result was a dispersion of solid particles in water, whose size was determined by dynamic light-scattering with the assistance of a Zetasizer™ nano S90 sold by the company MALVERN™ (table 2).

TABLE 2

| Test no. | Particle size (nm) |
|---|---|
| 3 | 100 |
| 5 | 30 |
| 8 | 150 |
| 9 | 110 |
| 10 | 130 |
| 11 | 115 |

These dispersions were stored for 21 days at ambient temperature and are stable. No sedimentation nor re-agglomerate were observed.

Finally, they were dried for 1 hour at 110° C. in an oven, which leads to the formation of granulates which enclose the oxaliplatin, encapsulated within polymer particles.

1. An aqueous formulation, comprising at least one organoplatinic compound and at least one associative water-soluble polymer, wherein said polymer is produced by a polymerization of:
(meth)acrylic acid;
at least one non-water-soluble monomer; and
at least one monomer represented by formula (I): 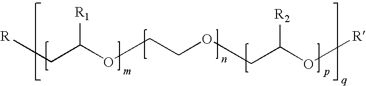
wherein
m, n, p and q are whole numbers and m, n, p are less than 150 and at least one of them is greater than 0,
R comprises a polymerizable vinylic function,
R₁ and R₂ are identical or different, and represent hydrogen atoms or alkyl groups comprising 1 to 4 carbon atoms,
R′ is a hydrophobic group comprising 14 to 32 carbon atoms.

2. The aqueous formulation according to claim 1, having a pH greater than 6.

3. The aqueous formulation according to claim 1, having a pH less than 6.

4. The aqueous formulation according to claim 1, comprising 0.1% to 30% by dry weight of the organoplatinic compound, relative to the dry weight of the associative water-soluble polymer.

5. The aqueous formulation according to claim 1, comprising 0.1 to 15% by weight of solids content in relation to the total weight of the aqueous formulation.

6. The aqueous formulation according to claim 1, wherein the organoplatinic compound is at least one selected from of the group consisting of cisplatin, carboplatin, and oxaliplatin.

7. The aqueous formulation according to claim 1, comprising at least one other anticancer agent selected from the group consisting of fluoro-uracil, S1, an association of vinblastin with bleomycin, an association of etoposide with bleomycin, and paclitaxel.

8. A granulate, comprising at least one organoplatinic compound and at least one associative water-soluble polymer, wherein the at least one associative water-soluble polymer is produced by a polymerization of:
(meth)acrylic acid;
at least one non-water-soluble monomer; and
at least one monomer represented by formula (I): 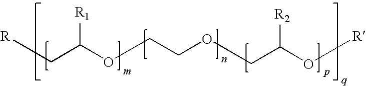
wherein
m, n, p and q are whole numbers and m, n, p are less than 150 and at least one of them is greater than 0;
R comprises a polymerizable vinylic function;
R₁ and R₂ are identical or different, and represent hydrogen atoms or alkyl groups comprising 1 to 4 carbon atoms,
R′ is a hydrophobic group comprising 14 to 32 carbon atoms.

9. A granulate according to claim 8, comprising less than 5% by weight of water, as measured by a differential scale after evaporation in an oven at 110° C. for 1 hour.

10. A granulate according to claim 8, comprising 0.1% to 30% by dry weight of the organoplatinic compound, relative to the dry weight of the at least one associative water-soluble polymer.

11. A granulate according to claim 8, wherein the organoplatinic compound is at least one selected from the group consisting of cisplatin, carboplatin, and oxaliplatin.

12. A granulate according to claim 8, comprising at least one other anticancer agent selected from the group consisting of fluoro-uracil, S1, an association of vinblastin with bleomycin, an association of etoposide with bleomycin, and paclitaxel.

13. A granulate according to claim 8, comprising at least one coating agent selected from the group consisting of a cellulosic polymer, and a copolymer of (meth)acrylic acid with an acrylic ester.

14. A granulate according to claim 8, comprising a lubricating agent.

15. A granulate according to claim 8, comprising a plastifying agent.

16. A granulate according to claim 8, comprising a surface-active agent.

17. A pharmaceutical preparation comprising an aqueous formulation according to claim 1.

18. The method of producing an orally administered polychemotherapeutic medication, comprising adding an aqueous formulation according to claim 1 to an orally administered polychemotherapy medication.

19. A pharmaceutical preparation comprising a granulate according to claim 8.

20. The method of producing an orally administered polychemotherapeutic medication, comprising adding a granulate according claims 8 to an orally administered polychemotherapy medication.
